(19) United States
(12) Patent Application Publication
(10) Pub. No.: US 2016/0082187 A1
(43) Pub. Date: Mar. 24, 2016

(54) DECISIONS SUPPORT FOR PATIENTS WITH DIABETES

(71) Applicant: Animas Corporation, West Chester, PA (US)

(72) Inventors: Thomas SCHAIBLE, Phoenixville, PA (US); Thomas MCCANN, Pottstown, PA (US); Jorge CAPURRO, West Chester, PA (US)

(21) Appl. No.: 14/494,018

(22) Filed: Sep. 23, 2014

(51) Int. Cl. A61M 5/172; A61B 5/00; A61B 5/145; A61M 5/142

(52) U.S. Cl. CPC A61M 5/1723; A61M 5/14244; A61B 5/6801; A61B 5/14532; A61M 2005/1726; A61M 2005/14208; A61M 2205/52; A61M 2205/502; A61M 2205/3584; A61M 2205/3592; A61M 2230/201

(57) ABSTRACT

A decision support system includes a measurement device configured to continuously measure a physiological parameter of a patient. An insulin delivery device provides insulin to the patient per an initial basal profile and the parameter measurements. A storage device holds historical data of insulin delivery to the patient. A processor determines deviations of the delivery of insulin from the basal profile for one or more time period(s) using the historical data, computes a respective first basal-profile adjustment for each of the one or more time period(s) using the determined deviations, and annunciates the computed first basal-profile adjustment(s). A method of recommending a basal-rate adjustment includes measuring the parameter, infusing the patient with insulin and storing the historical data, determining the deviations from the basal profile, computing the first basal-profile adjustments, and annunciating the computed first basal-profile adjustment(s).

### TECHNICAL FIELD

This application relates generally to the field of electronic systems for monitoring biological properties of a patient's body, and more specifically to medical monitoring systems.

### BACKGROUND

Diabetes mellitus is a chronic metabolic disorder caused by an inability of the pancreas to produce sufficient amounts of the hormone insulin, resulting in the decreased ability of the body to metabolize glucose. This failure leads to hyperglycemia, i.e. the presence of an excessive amount of glucose in the blood plasma. Persistent hyperglycemia and hypoinsulinemia have been associated with a variety of serious symptoms and life-threatening long-term complications such as dehydration, ketoacidosis, diabetic coma, cardiovascular diseases, chronic renal failure, retinal damage and nerve damages with the risk of amputation of extremities. Because restoration of endogenous insulin production is not yet possible, a permanent therapy is necessary which provides constant glycemic control in order to always maintain the level of blood glucose (BG) within normal limits. Such glycemic control is achieved by regularly supplying external insulin to the body of the patient to thereby reduce the elevated levels of blood glucose.

External biologic agents such as, for example, insulin or its analogs, can be administered as multiple daily injections of a mixture of rapid and intermediate-acting drugs via a hypodermic syringe. Improved glycemic control can be achieved by the so-called “intensive hormone” therapy which is based on multiple daily injections, including one or two injections per day of a long acting hormone for providing basal hormone and additional injections of rapidly acting hormone before each meal in an amount proportional to the size of the meal. Although traditional syringes have at least partly been replaced by insulin pens, the frequent injections are nevertheless very inconvenient for the patient, particularly those who are incapable of reliably self-administering injections. For some patients, substantial improvements in diabetes therapy have been achieved by the development of drug delivery devices, such as pumps and other insulin delivery or infusion systems, that relieve the patient of the need for syringes or drug pens and the need to administer multiple daily injections. Drug delivery devices can be constructed as an implantable device for subcutaneous arrangement or can be constructed as an external device with an infusion set for subcutaneous infusion to the patient via the transcutaneous insertion of a catheter, cannula or a transdermal drug transport, such as through a patch.

Blood or interstitial glucose monitoring can be used to achieve acceptable glycemic control. The determination of blood glucose concentration can be performed by means of an episodic measuring device, such as a hand-held electronic blood-glucose meter, that receives blood samples on enzyme-based test strips and calculates the blood glucose value based on an electrochemical reaction of the blood and the enzyme. An example of a handheld glucose meter/controller unit is the ONETOUCH PING™ from JOHNSON & JOHNSON®. Continuous glucose monitoring (CGM) using a sensor inserted into or implanted in the body can also be used. A combination of a CGM and a drug delivery device can be used to provide closed loop control of the insulin(s) being infused into the diabetic patients. To allow for closed-loop control of the infused insulins, proportional-integral-derivative (“PID”) controllers and model predictive controllers (MPC) have been used. The term “continuous” includes unceasing monitoring as well as frequent sampling. Exemplary CGM sensors generally sample glucose on a regular time scale, e.g., once per five minutes. Closed-loop control updates can be performed, e.g., in the time intervals between glucose measurements.

Drug delivery devices generally provide insulin at a “basal rate,” i.e., provide a certain amount of insulin every few minutes in a pre-programmed, daily pattern. Some drug delivery devices permit the user to manually request that a “bolus,” a specified amount of insulin, be delivered at a specified time. For example, before a meal, the user can request a bolus of additional insulin be delivered to process the glucose produced by digestion of the meal (a “carbohydrate correction bolus”). In another example, during a hyperglycemic excursion from a target blood-glucose range, the user can request a bolus to reduce blood sugar (a “glucose correction bolus”). Correction bolus amounts can be determined using an insulin-carbohydrate ratio (“I:C”) for carbohydrate correction boluses, and an insulin sensitivity factor (“ISF”) for glucose correction boluses. As used herein, the term “parameter” (or “parameters”) can refer to any or all of one or more basal rate(s), I:C value(s), or ISF value(s).

Parameters are generally set by a “titration” process. A patient's doctor selects initial values based on height, weight, or other factors, together with tables of statistical data. The patient then uses the pump and monitors blood glucose for a period of time, e.g., two weeks to three months. At the end of the period, the doctor reviews blood-glucose measurements and data on the operation of the pump during the period and determines adjustments to basal rates, I:C, or ISF. Adjustments can apply to an entire daily cycle or only part of a day (e.g., morning or night-time). In an example, if morning fasting glucose has consistently tested high during the period, the doctor can increase the basal rate during the overnight hours. This titration process is iterative and can be very time-consuming. Moreover, over the course of a long period such as three months, the patient's physiology can change, possibly decreasing the quality of care provided by the selected parameters. Moreover, the amount of data to be reviewed when the patient visits the doctor for updated parameters can be significant, requiring the doctor to spend considerable time reviewing the parameters.

As used herein, the term “dose period” or “scheduled dose period” refers to a period of time over which doses of insulin or other drugs, or the parameters used in determining the doses, are constant (barring boluses or other user actions). The term “long cycle” refers to a recurring pattern of dose periods. In an example, the dose period is hourly and the long cycle is daily. This example applies to an insulin delivery device that can deliver a (possibly different) amount of basal insulin every hour of the day, but the amount of basal insulin delivered, e.g., from 8 am to 9 am is the same each day. Such a device can store 24 basal insulin dose rates (U/hr) in a memory. In another example, the dose period is every three hours and the long cycle is 56 dose periods. This provides a selected (possibly unique) basal dose for each three-hour block throughout a week, after which the long cycle of 56 dose periods repeats. In still other examples, the dose period is 15 minutes or five minutes. The term “infusion period” refers to a period of time during which a selected amount of insulin is infused. For example, if a dosage of 3 U is to be applied over a one-hour dose period, the infusion period can be ten minutes and 0.5 U of insulin can be supplied to the patient in each of the six infusion periods in the hour.

### SUMMARY OF THE DISCLOSURE

In one embodiment, therefore, a decision support system for a patient has been devised. The system may include the following components:
a) a measurement device configured to continuously measure a physiological parameter of the patient;
b) an insulin delivery device configured to provide insulin to the patient according to an initial basal profile and the continuous measurements of the physiological parameter;
c) a storage device holding historical data of insulin delivery to the patient by the insulin delivery device; and
d) a processor coupled to the storage device, the processor being configured to:
   i) determine deviations of the delivery of insulin from the basal profile for one or more time period(s) using the historical data;
   ii) compute a respective first basal-profile adjustment for each of the one or more time period(s) using the determined deviations; and
   iii) annunciate the computed first basal-profile adjustment(s).

In another embodiment, a method of recommending a basal-rate adjustment for an insulin-delivery system is provided. The method can be achieved by:
continuously measuring a physiological parameter of a patient;
repeatedly infusing the patient with insulin according to an initial basal profile and the continuous physiological parameter measurements;
storing historical data of the delivery of insulin;
using a processor, automatically determining deviations of the delivery of insulin from the basal profile for one or more time period(s) using the stored historical data;
using the processor, automatically computing a respective first basal-profile adjustment for each of the time period(s) using the determined deviations; and
using the processor, automatically annunciating the computed first basal-profile adjustment(s).

Each of these embodiments, exemplary of the present invention, can provide improved determination and recommendation of adjustments to increase a patient's quality of care.

Accordingly, in any of the embodiments described earlier, the following features may also be utilized in various combinations with the previously disclosed embodiments. For example, the system can include the processor configured to process data for a plurality of time periods, to determine whether or not at least one of the plurality of time periods has deviations that are significantly different than an overall deviation of the plurality of time periods using a chi-squared (χ²) test; and to determine a single first basal-profile adjustment for at least two different ones of the plurality of time periods if the deviations for the at least one of the plurality of time periods are not significantly different than the overall deviation. The processor can be further adapted to adjust the initial basal profile based upon the computed first basal-profile adjustment(s). The system can include a display and the processor can be configured to annunciate the computed first basal-profile adjustment(s) by presenting a visual indication thereof on the display. Each first basal-profile adjustment can include a respective delivery rate and the visual indication can include textual representation(s) of the respective delivery rate(s). The system can include a user interface adapted to receive input and the processor can be further adapted to receive the historical data via the user interface and store the received historical data in the storage device. The historical data can include bolus data and the processor can be further configured to filter meal data out of the historical data using the bolus data. The measurement device can include a continuous glucose monitor and the measured physiological parameter can include blood glucose. The processor can be further configured to store blood glucose measurements of the patient and filter meal data out of the historical data using the stored measurements of the blood glucose. The processor can be further configured to store a plurality of the blood glucose measurements; determine deviations of blood glucose level from a stored aim range for one or more time period(s) using the stored blood glucose measurements; compute a respective second basal-profile adjustment for each of the one or more time period(s) using the determined deviations; and annunciate the respective second basal-profile adjustment(s). The processor can be further configured to filter meal data out of the stored blood glucose measurements using the historical data. The processor can be configured to determine the deviations of blood glucose level by determining the extent to which each of the stored blood glucose measurements is outside the stored aim range, and determining that the deviation for one of the time period(s) is zero if the stored measurements during that time period are within the stored aim range. The processor can be further configured to store a plurality of the blood glucose measurements for a selected time period; select two stored measurements using the historical data, the two stored measurements corresponding to a glucose correction bolus during the selected time period; determine a glucose effect of the glucose correction bolus using the selected stored measurements and a stored aim range; compute an adjustment to an insulin sensitivity factor for the selected time period using the determined glucose effect; and annunciate the computed adjustment to the insulin sensitivity factor. The storage device can hold an insulin-carbohydrate ratio and the processor can be further configured to store a plurality of the blood glucose measurements for a selected time period; select at least one stored measurement using the historical data, the selected at least one stored measurement corresponding to carbohydrate correction boluses during the selected time period; determine a respective deviation for each of the selected stored measurements with respect to a stored aim range; compute an adjustment to the insulin-carbohydrate ratio for the selected time period using the determined deviations and the insulin-carbohydrate ratio; and annunciate the respective adjustment to the insulin-carbohydrate ratio. The storage device can further hold a glucose-carbohydrate ratio and the processor can be further configured to compute the adjustment to the insulin-carbohydrate ratio using the stored glucose-carbohydrate ratio.

In various examples, the method can include measuring blood glucose as the physiological parameter. The method can include automatically, using the processor, storing a plurality of the blood glucose measurements; determining deviations of blood glucose level from a stored aim range for one or more time period(s) using the stored measurements; computing a respective second basal-profile adjustment for each of the time period(s) using the determined deviations; and annunciating the computed second basal-profile adjustment(s). The method can include automatically, using the processor, storing a plurality of the blood glucose measurements for a selected time period; selecting two stored measurements using the historical data, the two selected measurements corresponding to a glucose correction bolus during the selected time period; determining a glucose effect of the glucose correction bolus using the selected stored measurements and a stored aim range; computing an adjustment to an insulin sensitivity factor for the selected time period using the determined glucose effect; and annunciating the computed adjustment to the insulin sensitivity factor. The method can include automatically, using the processor, storing a plurality of the blood glucose measurements for a selected time period; selecting at least one of the stored measurements using the historical data, the at least one selected measurement corresponding to carbohydrate correction boluses during the selected time period; determining a respective deviation for each selected stored measurements with respect to a stored aim range; computing an adjustment to the insulin-carbohydrate ratio for the selected time period using the determined deviations and the insulin-carbohydrate ratio; and annunciating the computed adjustment to the insulin-carbohydrate ratio.

In the aforementioned aspects of the disclosure, the steps of measuring, infusing, storing, determining, computing, annunciating, storing blood glucose measurements, determining deviations of blood glucose level, computing second adjustments, annunciating second adjustments, storing, selecting, determining glucose effect, computing adjustment, annunciating adjustment, storing, selecting, determining, computing, and annunciating may be performed be an electronic circuit or a processor. These steps may also be implemented as executable instructions stored on a computer readable medium; the instructions, when executed by a computer may perform the steps of any one of the aforementioned methods.

In additional aspects of the disclosure, there are computer readable media, each medium comprising executable instructions, which, when executed by a computer, perform the steps of any one of the aforementioned methods.

In additional aspects of the disclosure, there are devices, such as test meters or analyte testing devices, each device or meter comprising an electronic circuit or processor configured to perform the steps of any one of the aforementioned methods.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention. For the sake of clarity, like reference numerals herein represent like elements.

FIG. 1 illustrates an exemplary glucose-monitoring and insulin delivery system and related components;

FIG. 2 shows an exemplary decision-support system for a patient and related components; and

FIGS. 3A-3B are a flowchart illustrating exemplary methods for recommending adjustments.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention or the attached claims.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values not at least ±10% of the recited value, e.g. “about 90%” may refer to the range of values from 81% to 99%. Throughout this disclosure, blood glucose values are given in mg/dL. Corresponding values in mmol/L can be calculated and used in any aspect described herein.

Throughout this disclosure, the terms “patient” and “subject” are used interchangeably. These terms can refer to any human or animal subject and are not intended to limit the systems or methods to human use, although use of the subject invention in a human patient represents a preferred embodiment. Furthermore, in this disclosure, the term “user” can refer to a patient using a glucose measuring or drug delivery device or to another person (e.g., a parent or guardian, nursing staff member, home care employee, or other caretaker) using such a device. The term “healthcare provider” or “HCP” refers generally to doctors, nurses, and individuals other than the patient that provide health care services to the patient. The term “drug” may include hormones, biologically active materials, pharmaceuticals or other chemicals that cause a biological response (e.g., a glycemic response) in the body of a user or patient.

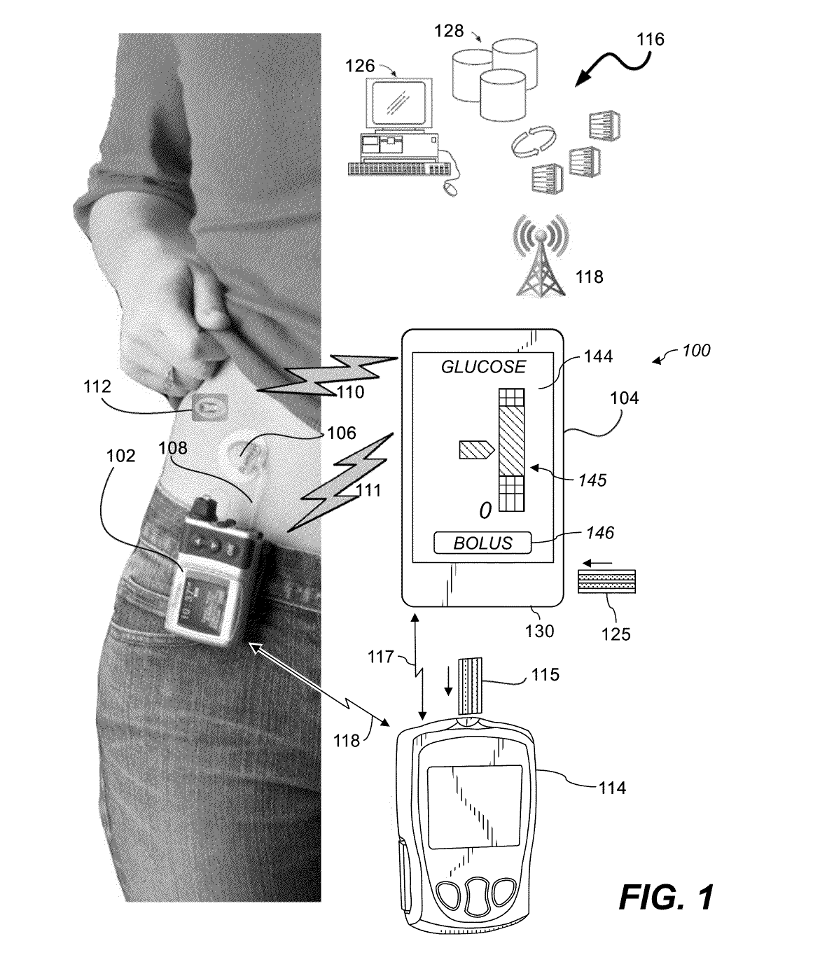

FIG. 1 illustrates an exemplary glucose-monitoring and insulin delivery system 100, e.g., an artificial pancreas. In this specific example, an insulin delivery device 102 is connected to an infusion set 106 via flexible tubing 108 and is controlled, e.g., by a controller 104. Various embodiments of the invention can also be used with injections via syringe or insulin pen instead of or in addition to infusion via the insulin delivery device 102. The controller 104 or insulin delivery device 102 can communicate with a continuous glucose monitoring (CGM) sensor 112. In an example, the controller 104, insulin delivery device 102, and CGM sensor 112 cooperate to attempt to maintain a user's blood glucose level within an aim range, e.g., 70-130 mg/dL, and more specifically to try to drive the user's blood glucose level to a target, e.g., 100 mg/dL.

The insulin delivery device 102 is configured to transmit and receive data to and from the controller 104 by, for example, a radio frequency (RF) communications link 111. In one embodiment, the insulin delivery device 102 is an insulin infusion device and the controller 104 is a hand-held portable controller. In such an embodiment, data transmitted from the insulin delivery device 102 to the controller 104 may include information such as, for example, insulin delivery data, blood glucose (BG) information, basal, bolus, insulin to carbohydrates ratio or insulin sensitivity factor. The controller 104 can be configured to include a closed-loop controller that has been programmed to receive continuous glucose readings from the CGM sensor 112 via a radio frequency (RF) communications link 110. The CGM sensor 112 can measure glucose levels of interstitial fluid in the body, determine corresponding blood glucose levels, and provide the BG levels to the controller 104. The CGM sensor 112 can also or alternatively provide data representative of or proportional to blood-glucose values directly to the insulin delivery device 102 via a radio frequency (RF) communications link 113 or a wired connection such as a Universal Serial Bus (USB) cable.

Data transmitted from the controller 104 to the insulin delivery device 102 may include glucose test results and a food database to allow the insulin delivery device 102 to calculate the amount of insulin to be delivered by the insulin delivery device 102. Alternatively, the controller 104 may perform basal dosing or bolus calculation and send the results of such calculations to the insulin delivery device. A glucose meter 114 (here, an episodic blood-glucose meter), alone or in conjunction with the CGM sensor 112, provides data to either or both of the controller 104 and insulin delivery device 102, e.g., via a radio frequency (RF) communications link 117. The glucose meter 114 can measure a fluid sample placed on a test strip 115. The two hatched areas on the test strip 115 graphically represent two electrodes, as is discussed below. The glucose meter 114 can include a display or other interface to present information, or can present information only via the controller 104.

For purposes of this embodiment, the test strip 115 is defined by a planar substrate over which are disposed the electrodes (shown hatched; formed of, e.g., sputtered gold or palladium) and corresponding electrical contact pads (not shown). The electrodes can be disposed on opposing sides of a sample-receiving chamber, above and below the sample-receiving chamber, or in other configurations. The exemplary test strip 115 includes a working electrode formed by sputtering a palladium (Pd) coating on a polyester substrate and a reference electrode formed by sputtering gold (Au) on the polyester substrate. A dry reagent layer can be used and can include a buffer and a mediator. Various enzymes in the reagent layer or elsewhere in the sample-receiving chamber can assist in transducing the analyte (e.g., glucose) in the fluid sample (e.g., blood, interstitial fluid, or control solution) into a current, potential, or other quantity that can be measured electrically. Exemplary enzymes include glucose oxidase, glucose dehydrogenase (GDH) based on a pyrroloquinoline quinone co-factor, and GDH based on a nicotinamide adenine dinucleotide co-factor. Exemplary glucose sensors and associated components are shown and described in U.S. Pat. Nos. 6,179,979, 8,163,162, and 6,444,115, which are incorporated by reference herein in their entireties.

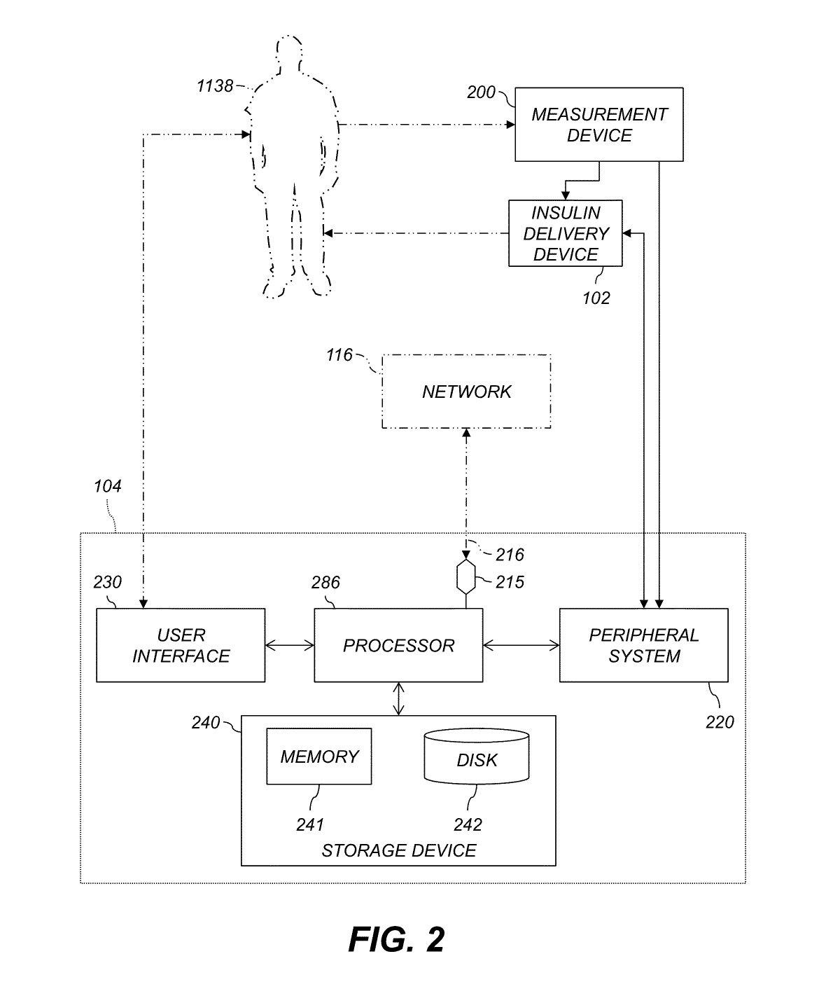

The controller 104 can present information and receive commands via a touchscreen 144 or other devices discussed below with reference to a user interface 230, FIG. 2. In the example shown, the controller 104 is presenting tape and numeric indicators representing a recent blood-glucose measurement (“120 mg/dL”). An exemplary tape indicator 145 has (from top to bottom) yellow, green, yellow, and red sections indicating various ranges of blood glucose, and a pointer representing the recent measurement. The measurement is in the green range so is colored green. The controller 104 is also presenting a “BOLUS” soft key 146 on the touchscreen 144. The user can press this soft key to request a bolus of insulin.

The controller 104, the insulin delivery device 102, and the CGM sensor 112 can be integrated into multi-function units in any combination. For example, the controller 104 can be integrated with the insulin delivery device 102 to form a combined device with a single housing. Infusion, sensing, and controlling functions can also be integrated into a monolithic artificial pancreas. In various embodiments, the controller 104 is combined with the glucose meter 114 into an integrated monolithic device having a housing 130. Such an integrated monolithic device can receive a test strip 125. In other embodiments, the controller 104 and the glucose meter 114 are two separable devices that are dockable with each other to form an integrated device. Each of the devices 102, 104, and 114 can include a suitable processor or microcontroller (not shown for brevity) programmed to carry out various functionalities. Examples of microcontrollers that can be used are discussed below with reference to a processor 286, FIG. 2.

The insulin delivery device 102 or the controller 104 can also be configured for bi-directional communication with a network 116 through, for example, a radio frequency communications link 118. One or more server(s) 126 or storage device(s) 128 can be communicatively connected to the controller 104 via the network 116. In an example, the insulin delivery device 102 communicates with a personal computer (e.g., the controller 104) via BLUETOOTH. The controller 104 and the network 116 can be configured for bi-directional wired communication through, for example, a telephone land based communication network. The controller 104 can include a smartphone, electronic tablet, or personal computer.

The insulin delivery device 102 can include any or all of: electronic signal processing components including a central processing unit and memory elements for storing control programs and operation data, a radio frequency module (not shown) for sending and receiving communication signals (e.g., messages) to and from the controller 104, a display for providing operational information to the user, a plurality of navigational buttons for the user to input information, a battery for providing power to the system, an alarm (e.g., visual, auditory or tactile) for providing feedback to the user, a vibrator for providing feedback to the user, and an insulin delivery mechanism (e.g., a drug pump and drive mechanism) for forcing a insulin from a insulin reservoir (e.g., a insulin cartridge) through a side port connected via the flexible tubing 108 to an infusion set 106 and into the body of the user.

Various glucose management systems include an episodic glucose sensor (e.g., the glucose meter 114) and an infusion pump. An example of such a system is the ONETOUCH PING Glucose Management System manufactured by the Animas Corporation. The “ezBG” feature of this system computes an amount of insulin to be delivered by the infusion pump using the results of an episodic glucose measurement. Another example of a glucose management system is the ANIMAS VIBE™ insulin pump, which communicates with a DEXCOM G4™ CGM system manufactured by the DexCom Corporation. Interfaces can be provided to connect these components. Closed-loop control algorithms can be programmed in, e.g., the MATLAB™ language to regulate the rate of insulin delivery based on the glucose level of the patient, historical glucose measurement and anticipated future glucose trends, and patient specific information.

FIG. 2 shows an exemplary decision support system for a patient, including data-processing components for analyzing data and performing other analyses and functions described herein, and related components. A patient 1138 and a network 116 are not part of the system but are shown for purposes of context. The controller 104 can communicate with a measurement device 200 (e.g., a CGM sensor 112, FIG. 1) or the insulin delivery device 102, e.g., via a peripheral system 220. The controller 104 can also communicate with the network 116, e.g., a cellular telephone data network or the Internet. The controller 104 can also include the user interface 230 and a storage device 240 communicatively connected to the processor 286, as discussed below. The processor 286, upon receipt of data from a device in the peripheral system 220, can store that data in the storage device 240.

The measurement device 200 is configured to continuously measure a physiological parameter of the patient. In an example, the measurement device 200 comprises a continuous glucose monitor and the measured physiological parameter comprises blood glucose. The processor 286 in the controller 104 can receive glucose data from the measurement device 200 (the CGM sensor 112, or the glucose meter 114 using the test strip 115) and provide control signals to the insulin delivery device 102 to deliver insulin to the patient 1138, as discussed below. The insulin delivery device 102 can also or alternatively receive the glucose data from the measurement device 200 and adjust the insulin to be delivered to the patient 1138.

In various aspects, the insulin delivery device 102 is configured to provide insulin to the patient 1138 according to an initial basal profile and the continuous measurements of the physiological parameter. The initial basal profile can include data of respective dosage(s) for one or more dose period(s). The dosage can be, e.g., in units of insulin (U) per hour or per infusion.

The insulin delivery device 102 in this example receives the continuous measurements of the physical parameter (e.g., glucose data) from the measurement device 200 and operates a closed-loop control law, e.g., using an embedded processor (not shown; e.g., a processor similar to the processor 286) in the insulin delivery device 102. In this way, the insulin delivery device can adjust for at least some excursions in blood glucose. For example, during a hyperglycemic excursion, the control law operates to increase the amount of insulin delivered to the patient 1138 above the amount specified in the initial basal profile. As used herein, the term “force” refers to the difference between the amount of insulin delivered in an infusion period or dose period and the amount of insulin specified by the initial basal profile for that infusion period or dose period. During a hyperglycemic excursion, the force will generally be positive. During a hypoglycemic excursion, the force will generally be negative.

According to this exemplary embodiment, the storage device 240 holds historical data of insulin delivery to the patient by the insulin delivery device. The historical data can be received from the insulin delivery device 102 via the peripheral system 220, discussed below. In an example, the storage device 240 includes a memory 241, e.g., a random-access memory, and a disk 242, e.g., a tangible computer-readable storage device such as a hard drive or a solid-state flash drive. The memory 241 or the disk 242 can store data used by running programs. For example, the historical data of insulin delivery can be stored in the memory 241 or on the disk 242.

The processor 286 can be coupled to the storage device 240 and configured to perform various functions described herein. For example, the processor 286 can be configured to determine deviations of the delivery of insulin from the basal profile for one or more dose period(s) time period(s) in a day using the historical data.

In an example, the historical data include U/hr values for each dose period over the course of a long cycle. The processor 286 retrieves the historical data for a plurality of long cycles, e.g., about 30 long cycles, from the storage device 240. The processor 286 also retrieves the initial basal profile from the storage device 240. For each dose period, the processor 286 computes, as a deviation for that dose period, the average difference between the historical dosage for that dose period and the initial basal profile for that dose period.

In various embodiments, the historical data include n U/hr values (n≧1) for each five- or 15-minute interval (or other interval length) in a dose period. These values represent the basal rate recommended by the closed-loop algorithm. In various aspects, if the amount of insulin delivered in a particular dose was adjusted (e.g., limited) because of an algorithm to reduce the probability of insulin-induced hypoglycemic excursions, the historical data can include the adjusted amount, the pre-adjustment amount, or the force (adjusted minus pre-adjustment). The forces, i.e., the differences between each of the n values and the initial basal rate for the dose period, are determined and divided by n to determine the deviation.

In various examples, data are collected for 30 long cycles, e.g., 30 days, or 14 long cycles or more, or at least 7 long cycles. In various aspects, if the user requests recommendations based on only seven days' historical data (or another selected threshold on the number of long cycles), the processor 286 prompts the user to confirm that those days are representative of the patient's typical activity and not, e.g., a vacation week with unusual mealtimes.

The processor 286 is further configured to compute a respective first basal-profile adjustment for each of the dose period(s) using the determined deviations. It should be noted that the processor 286 can leave the doses unchanged for one or more dose period(s), in various examples. In an example, the processor 286 determines that the respective first basal-profile adjustment is zero if the deviation has a magnitude less than a selected threshold, e.g., within ±0.5 U/hr. The processor 286 determines that the adjustment is the deviation if the deviation has a magnitude over the threshold, e.g., not within ±0.5 U/hr. The threshold can be selected based on the granularity of dosing provided by the insulin delivery device 102.

In at least one embodiment, the processor 286 is configured to annunciate the computed first basal-profile adjustment(s). The processor 286 can, e.g., present a human-perceptible indication of the adjustment(s) via the user interface 230. This advantageously provides additional information to doctors or patients and assists doctors in concentrating on clinically-relevant deviations. To further assist doctors, the processor 286 can present fewer than all of the determined adjustment(s), e.g., by filtering out adjustments having a magnitude lower than a doctor-selected or other threshold.

The user interface 230 can include a display device, a touchscreen, a processor-accessible memory, or any device or combination of devices to which data is output by the processor 286. In this regard, if the user interface 230 includes a processor-accessible memory, such memory can be part of the storage device 240 even though the user interface 230 and the storage device 240 are shown separately in FIG. 6. For example, the user interface 230 can include one or more touchscreen(s), speaker(s), buzzer(s), vibrator(s), button(s), switch(es), jack(s), plug(s), or network connection(s).

In various aspects, the processor 286 is configured to annunciate score(s) for one or more of the adjustment(s) instead of or in addition to the numerical value(s) of those adjustment(s). The processor 286 can be configured to determine each score using a respective one of the adjustment(s). In an example, a score is annunciated for each dose period. Annunciating scores rather than adjustments advantageously permits healthcare providers to concentrate on dose periods for which adjustments might, e.g., provide a therapeutic benefit.

In an example, the scores can be modeled on the academic scoring system in use in the patient's country, e.g., A, B, C, D, F (best to worst) in the United States or 1-7 in Scotland. Other scoring systems can be used, e.g., the ◯, Δ, X (best to worst) system used in Japan. In another example, colors, shades of gray, or combinations thereof can be used, e.g., green, yellow, red or white, gray, black (best to worst). The “worst” score can represent the adjustment the processor 286 deems most significant. The processor 286 can map between an adjustment and the corresponding score in a linear or nonlinear fashion, optionally with saturation and offset. For example, in an A-F scale, the A, B, C, D, and F scores can cover respective ranges of the adjustment arranged in a geometric progression, with A being the widest band. In a specific example, with a ratio of 1.47 and adjustments normalized from 0% (no adjustment) to 100% (a selected adjustment-amount limit), A can be 0%-37%, B 37%-63%, C 63%-80%, D 80%-92%, and F 92%-100%. The A score can also be the narrowest band. Scores can be presented in a chart of, e.g., the dose periods.

In various aspects, the processor 286 is configured to determine whether an adjustment should be made by applying a statistical test to the determined deviations. In an exemplary embodiment, the processor 286 retrieves the historical data dosage values for a plurality of dose periods and determines respective deltas by subtracting from each historical dosage for each dose period the initial basal profile for that dose period. The processor 286 then applies a chi-squared (χ²) test to determine whether the any one or more of the dose periods has deltas that are significantly different from the deltas for the others of the plurality of dose periods, e.g., at the 95% confidence level or another selected confidence level. If the deltas are significantly different for one or more of the dose period(s), the processor 286 can compute and annunciate the first basal-profile adjustments for the one or more of the dose period(s).

In various aspects, therefore, the one or more time period(s) include a plurality of time periods (e.g., dose periods). The processor 286 is further adapted to determine whether or not at least one of the plurality of time periods has deviations that are significantly different than an overall deviation of the plurality of time periods using a χ² test. This is discussed below. The processor 286 is yet further configured to determine a single first basal-profile adjustment for at least two different ones of the plurality of time periods if the deviations for the at least one of the plurality of time periods are not significantly different than the overall deviation. The χ² test indicates whether there are specific time periods, e.g., dose periods, on which attention might profitably be focused. If there is no such specific time period, the time periods as a whole may still be in need of adjustment. This is also discussed below.

In an example, for each hour of the day or other dose period, the processor 286 divides historical data values for a plurality of infusion periods depending on whether the force during that infusion period had a greater magnitude than a selected threshold, e.g., a force more negative than −0.5 U/hr or more positive than 0.5 U/hr was applied. For each dose period i, the infusion periods with such a magnitude of force are counted as Oᵢ₁, and the infusion periods without such a magnitude of force are counted as Oᵢ₂. The counts (O) are respective observed values. The total number M₁ of historical data values over a long cycle for which the magnitude of force was applied is then computed:

$${M}_{1} = {\sum }_{i = 1}^{I} {O}_{i 1}$$

where I is the number of dose periods in a long cycle. M₂ is computed similarly, using the Oᵢ₂ values. The numbers Nᵢ of readings for each dose period i are then computed, as is the total number of readings N in the long cycle:

$${N}_{i} = {O}_{i 1} + {O}_{i 2}$$

$$N = {\sum }_{i = 1}^{I} {N}_{i}$$

Expected values Eᵢⱼ are then computed:

$${E}_{i 1} = \frac{{N}_{i} {M}_{1}}{N}$$

for historical data for which the selected magnitude of force was applied, and

$${E}_{i 2} = \frac{{N}_{i} {M}_{2}}{N}$$

for historical data for which the selected magnitude of force was not applied. In these equations, N is the total number of samples, e.g., the number of times insulin was delivered, in the long cycle. Nᵢ is the total number of samples in each dose period. M₁ is the total number of samples across the long cycle in which force was applied beyond the threshold. The expected value Eᵢ₁ represents the likely number of times that force would be applied beyond the threshold if such force were equally likely to be applied every time a dose was infused (every infusion cycle). Such evenly-spread likelihood is referred to as an “equal distribution.”

The processor then computes a chi-squared (χ²) statistic using the O and E values as follows:

$${χ}^{2} = {\sum }_{j = 1}^{2} {\sum }_{i = 1}^{I} \frac{{( {O}_{ij} - {E}_{ij} )}^{2}}{{E}_{ij}} \text{:}$$

where j=1 refers to infusion periods with the magnitude of force and j=2 refers to infusion periods without the magnitude of force, and i is an index of the dose period in a long cycle. For hourly dose periods in a day, i ranges from 1 to 1=24. This is an example of Pearson's χ² test; other tests can also be used.

The χ² value is compared to a χ² distribution with an appropriate number of degrees of freedom (DOF) to determine significance. In the hourly/daily example, the number of DOF is 23. The probability that the computed χ² value corresponds to the equal distribution is determined from the χ² distribution. If the resulting probability is less than a selected confidence threshold (e.g., 0.05 or 0.01), the processor 286 determines that the long cycle includes dose period(s) that are statistically different from other dose period(s) in the long cycle. That is, at least one dose period during the tested long cycle has deltas or deviations that are different from the deltas for the other dose periods in a statistically significant way. If the χ² test indicates that there is not such a statistically significant difference between the observed values and the equal distribution, the processor 286 can determine that either the initial basal profile is correctly tracking the patient's physiology, or that deviations (and force) are relatively consistent across the long cycle.

If the χ² test indicates that at least some of the deviations are not consistent across the long cycle (e.g., probability less than 0.05), the processor 286 further determines a Z score for each dose period. The processor 286 then determines and annunciates adjustments for dose periods with a Z-value having a magnitude greater than a threshold, e.g., |Z|>2.0.

To compute the Z-score, the processor 286 first computes the standard error SEᵢ of each dose period i:

$${SE}_{i} = \sqrt{{E}_{i 1} ( 1 - \frac{1}{N} )}$$

The processor 286 can then compute the Z-score for each dose period i:

$${Z}_{i} = \frac{( {O}_{i 1} - {E}_{i 1} )}{{SE}_{i}}$$

The Z-value for time period i corresponds to the number of standard deviations away from the mean dose period i is, but uses values from a sample, not a statistical population.

Table 1 shows an exemplary χ² table for hourly dose periods and daily long cycles. Table 1 shows how the various quantities described above interrelate.

TABLE 1

| | | | Samples with | | | | |
|---|---|---|---|---|---|---|---|
| | Samples with | | Force Not | | | | |
| | Force Applied | | Applied | | | | |
| | Obs | Exp | Obs | Exp | Row Total | SE | Z |
| Hour 1 | O₁₁ | E₁₁ | O₁₂ | E₁₂ | N₁ | SE₁ | Z₁ |
| Hour 2 | O₂₁ | E₂₁ | O₂₂ | E₂₂ | N₂ | SE₂ | Z₂ |
| . . . | . . . | . . . | . . . | . . . | . . . | . . . | . . . |
| Hour i | Oᵢ₁ | Eᵢ₁ | Oᵢ₂ | Eᵢ₂ | Nᵢ | SEᵢ | Zᵢ |
| . . . | . . . | . . . | . . . | . . . | . . . | . . . | . . . |
| Hour I = 24 | O_{I1} | E_{I1} | O_{I2} | E_{I2} | N_{I} | SE_{I} | Z_{I} |

| | | |
|---|---|---|
| M₁ | M₂ | N |

If the χ² test indicates that the deviations are consistent across the long cycle (e.g., probability greater than 0.05), the processor 286 in at least one example determines a single first basal-profile adjustment for at least two different ones of the plurality of time periods. Since the deviations for the time periods are consistent, the adjustments can also be consistent.

In various embodiments, the processor 286 can be further adapted to adjust the initial basal profile based upon the computed first basal-profile adjustment(s). This adjustment can be done on a selected time granularity, e.g., once per long cycle or per selected number of long cycles; once per month; once per quarter; or at other intervals. This can advantageously improve the accuracy of insulin dosing for each specific patient 1138.

In various aspects, the user interface 230 includes a display such as the touchscreen 144, FIG. 1. The processor 286 can be configured to annunciate the computed first basal-profile adjustment(s) by presenting a visual indication thereof on the display. In an example, each first basal-profile adjustment includes a respective delivery rate in U/hr. The visual indication includes textual representation(s) of the respective delivery rate(s).

In at least one embodiment, the user interface 230 is adapted to receive input, e.g., from the patient 1138. The processor 286 can be further adapted to receive the historical data via the user interface 230 and store the received historical data in the storage device 240. For example, the patient 1138 can record pump levels on paper, and enter those into the controller 104 via the user interface 230. The insulin delivery device 102 can also store the historical data on a removable medium, e.g., a Flash drive, and the user interface 230 can receive that removable medium to be read by the processor 286.

The user interface 230 can include a mouse, a keyboard, another computer (connected, e.g., via a network or a null-modem cable), a microphone and speech processor or other device(s) for receiving voice commands, a camera and image processor or other device(s) for receiving visual commands, e.g., gestures, or any device or combination of devices from which data is input to the processor 286. In this regard, although the peripheral system 220 is shown separately from the user interface 230, the peripheral system 220 can be included as part of the user interface 230. In at least one embodiment, the user interface 230 can be operated by the patient 1138.

In an example, the historical data includes bolus data. The processor 286 can be further configured to filter meal data out of the historical data using the bolus data. For example, before a meal, the patient 1138 will often request a bolus to process the estimated carbohydrate content of the meal. Some controllers 104 or insulin delivery devices 102 permit the patient 1138 to indicate that a particular bolus is a preprandial (pre-meal) bolus. These indications are stored as part of the historical data. Accordingly, the historical data for, e.g., 1.5 hours to four hours after the meal bolus can be disregarded since glucose levels are not at the steady state the basal profile is designed to maintain. Other controllers 104 or insulin delivery devices 102 do not permit the patient 1138 to provide such an indication. However, preprandial boluses are often larger in mass or volume than correction boluses. A threshold can be stored in the storage device 240, and the processor 286 can compare bolus(es) indicated in the historical data to the threshold and disregard data after over-threshold boluses.

In various examples, the processor 286 is further configured to store blood glucose measurements of the patient and filter meal data out of the historical data using the stored measurements of the blood glucose. The processor 286 can receive the blood glucose measurements from the measurement device 200. In an example, the measurement device 200 is a CGM sensor testing approximately every five minutes. A hyperglycemic excursion lasting, e.g., more than 30 minutes, with an average increase in BG of 3 mg/dL/min, can indicate the onset of blood sugar increase due to a meal. Historical data following the meal, and optionally for a selected time before the meal, can be disregarded.

In various aspects, the processor 286 is further configured to store a plurality of the blood glucose measurements from the measurement device 200. The processor 286 then determines deviations of blood glucose level from a stored aim range for one or more dose period(s) using the stored blood glucose measurements. The dose period(s) used with respect to the historical data can be the same as, or different from, the does period(s) used with respect to the blood glucose measurements. The processor 286 computes a respective second basal-profile adjustment for each of the one or more dose period(s) using the determined deviations. For some dose period(s), the processor 286 can provide a second basal-profile adjustment of zero (i.e., unchanged). The processor 286 then annunciates at least some of the respective second basal-profile adjustment(s), e.g., via the user interface 230. The processor 286 can annunciate by presenting via the user interface 230 a human-perceptible indication of the second basal-profile adjustments.

The stored blood glucose measurements can cover, e.g., 30 days (or long cycles) with 1-2 glucose measurements per day (long cycle), or up to 90 days, or as few as 14 days. In an aspect, shorter coverage periods include more glucose measurements per long cycle, e.g., three tests for each of 14 days. Glucose measurements can be spread out over the course of a long cycle.

In an example, the deviation is the average extent (in mg/dL) by which blood glucose measurements are out of an aim glucose range over the dose period. The second basal-profile adjustment for a given dose period can be (in U/hr) the deviation for that dose period divided by the insulin sensitivity factor (ISF) for that dose period. In another example, the deviation is the difference between the target glucose and the average glucose for all readings during the dose period, over the course of, e.g., 30 days. The adjustment is that difference divided by ISF. The adjustment can be applied ahead of the time of the glucose measurement, e.g., one hour ahead. For example, the long cycle can be daily and the dose period can be hourly (numbered from 0 to 23). For a glucose measurement in dose period p, the adjustment can be applied to dose period p−1. If a glucose excursion extends over more than one dose period, the processor 286 can determine adjustments to more than one dose period.

In various aspects, the processor 286 is configured to determine the deviations of blood glucose level by determining the extent to which each of the stored blood glucose measurements is outside the stored aim range, as described above. The processor 286 is further configured to determine that the deviation for one of the time period(s) is zero if the stored measurements during that time period are within the stored aim range. This advantageously permits the basal rate to continue unadjusted as long as blood glucose is experiencing normal variation within the aim range, and reduces the effects of natural noise on the computation of the second basal-profile adjustments. The deviations can be positive or negative, and the corresponding adjustments can be negative or positive. This can advantageously assist in reducing the incidence of hyperglycemia or hypoglycemia, or providing a doctor information to do so.

The processor 286 can be further configured to filter meal data out of the stored blood glucose measurements using the historical data. For example, meals can be located using the historical data as described above. Examples include using bolus data; using data entered in a food tracking database, e.g., on an insulin pump or on the controller 104; and receiving input from the user's paper records or from a diabetes management system. Stored blood glucose measurements within the 1.5-4 hours after a meal can then be disregarded, or adjusted downward by, e.g., 50 mg/dL, to correct for the effects of ingested carbohydrates.

In various embodiments for computing the second basal-profile adjustments, the processor 286 is configured to store and use ISF values in its calculations. In general, parameters such as ISF and I:C can be used for various purposes. Various aspects described herein therefore provide devices and methods for annunciating adjustments to the parameters. ISF can be used by a bolus calculator in determining a bolus amount to move a patient's blood sugar from out-of-range to target. Such calculators can be particularly useful to people who test more frequently, e.g., patients with Type 1 diabetes who test at least three times per day.

In various aspects, the processor 286 is further configured to store a plurality of the blood glucose measurements for a selected time period, e.g., a full day or other long cycle, or a dose period. In an example in which the time period is a full day, if the patient takes glucose measurements using an episodic meter at 6 am, 2 pm, and 10 pm, the processor 286 can store the 14 (or more) 6 am measurements, the ≧14 2 pm measurements, and the ≧14 10 pm measurements. In an example using CGM data and a one-hour time period, the processor stores 12 glucose measurements per hour (at five-minute intervals) and stores up to all 12 measurements for each hour of the day for 14 days.

The processor 286 is configured to select two stored measurements using the historical data, the two stored measurements corresponding to a glucose correction bolus during the selected time period. The two measurements include a “before” measurement and an “after” measurement. The “before” measurement can be the most recent BG measurement before the bolus or other BG measurement used in computing the bolus amount. The “after” measurement can be a BG measurement taken between, e.g., 1.5 and 4 hours after the bolus, or a median or average of multiple BG measurements between those times. Glucose correction boluses can be located in the historical data as described above. The processor 286 can also select multiple pairs of “before” and “after” measurements and perform processing described below for each pair. In various examples, the processor 286 selects and uses 20 pairs, or at least 20 pairs, of measurements in the given time period.

The processor 286 is further configured to determine a glucose effect of the glucose correction bolus using the selected stored measurements and a stored aim range. This can be done by adjusting the “after” value to be equal to a selected target if the “after” measurement is within the selected aim range. The (possibly adjusted) “after” value is then subtracted from the “before” value to determine the drop in insulin resulting from the bolus. This drop is the glucose effect (Δmg/dL). In various aspects using multiple pairs of “before” and “after” measurements, the processor 286 computes a respective glucose effect for each pair of measurements. In an example, the American Diabetes Association recommends preprandial glucose of 90-130 mg/dL. The selected target can thus be the midpoint of that range, 110 mg/dL. The “before” and “after” values can be in the normal, hypoglycemic, or hyperglycemic ranges.

The processor 286 is still further configured to compute an adjustment to an insulin sensitivity factor (ISF) for the selected time period using the determined glucose effect, and to annunciate the computed adjustment to the insulin sensitivity factor. The annunciating can be via the user interface 230. As above, there can be time periods for which an adjustment is not computed or not annunciated, e.g., periods for which the adjustment is smaller than the resolution of the ISF value stored in the insulin delivery device 102 (e.g., 1.0 U/mg/dL). The processor 286 can compute the adjustment by dividing the size of the bolus (U) by the determined glucose effect (Δmg/dL). ISF can be expressed as U/(Δmg/dL) or as (Δmg/dL)/U; either can be computed by the processor 286. In various aspects using multiple pairs of “before” and “after” measurements, the processor 286 computes a respective adjustment for each pair of measurements, then averages the respective adjustments to determine the adjustment to the ISF. This averaging can mitigate the effect on ISF of user errors in manual data recording or data entry.

In various aspects, the processor 286 is further configured to automatically apply the computed adjustment to the stored insulin sensitivity factor(s) corresponding to the time period. The ISF can be updated for the time at which the bolus was delivered.

Another parameter is insulin-carbohydrate ratio (I:C), which can be stored in the storage device 240. The patient, via the controller 104, can use I:C ratio to compute the appropriate amount of insulin for a pre- or postprandial bolus. In various aspects, the processor 286 is configured to annunciate an adjustment to I:C. The processor 286 is configured to store a plurality of the blood glucose measurements for a selected time period, as discussed above. The processor 286 is further configured to select a stored measurement using the historical data, the selected measurement corresponding to a carbohydrate correction bolus during the selected time period. For example, the selected measurement can be a measurement taken between 1.5 and 4 hours after such a bolus.

The processor 286 is configured to determine a respective deviation for each of the selected stored measurements with respect to a stored aim range. In an example, if the respective measurement of glucose is within the aim range, that measurement is adjusted to be equal to the target. Each deviation is then determined as (adjusted) measurement minus target BG.

In an example, the storage device holds a glucose-carbohydrate (“G:C”) ratio. The G:C ratio represents the typical effect on blood glucose of ingesting a unit amount of carbohydrate. In an example, the G:C ratio is 5 mg/dL per gram CHO. The G:C ratio can be obtained from clinical studies and can be varied per patient. Typical G:C ratios are between 5 and 10 mg/dL/g(CHO), but ratios outside that range can also be used.

In this example, the processor 286 computes an adjustment to the insulin-carbohydrate ratio for the selected time period using the determined deviations and the insulin-carbohydrate ratio, and the G:C ratio. The processor 286 then annunciates, e.g., via the user interface 230, the respective adjustment to the insulin-carbohydrate ratio. The processor 286 can compute an adjustment for all or fewer than all time periods in, e.g., a long cycle or other time range, and the time period(s) can be the same as those used in determining adjustments for basal rates and ISF, or can be different. In this example, the adjustment can be computed by taking the average of all the deviations and dividing by the G:C ratio. In various aspects, adjustments can be computed only if the average deviation has more than a selected magnitude, e.g., 30 mg/dL.

In various aspects, the processor 286 is further configured to automatically update the I:C ratio stored in the storage device 240.

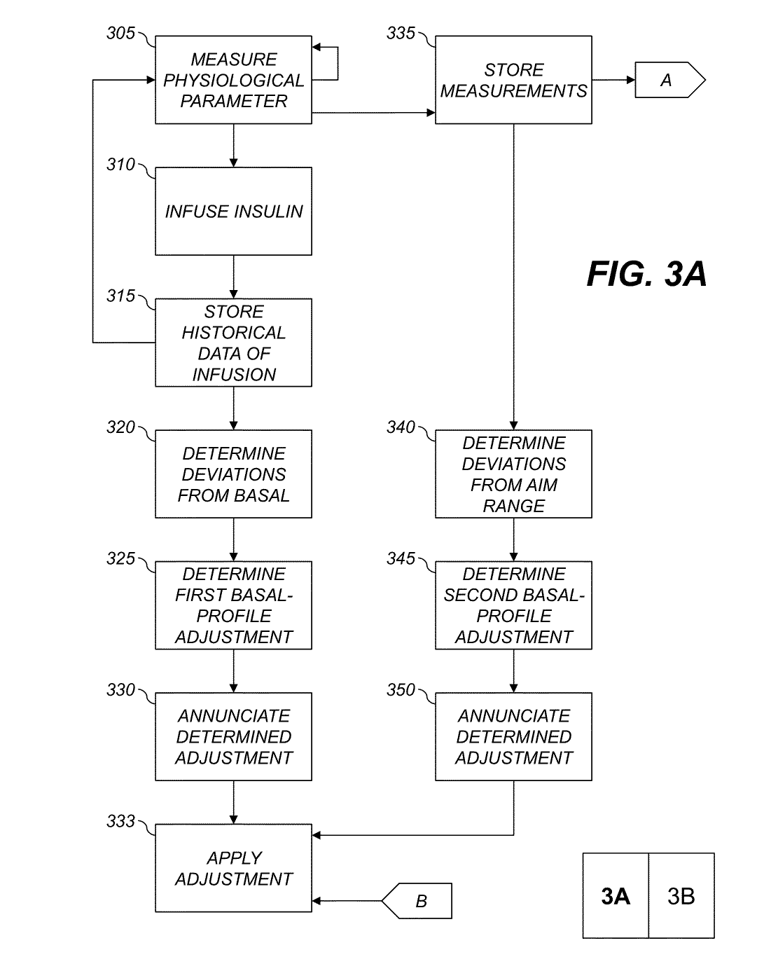

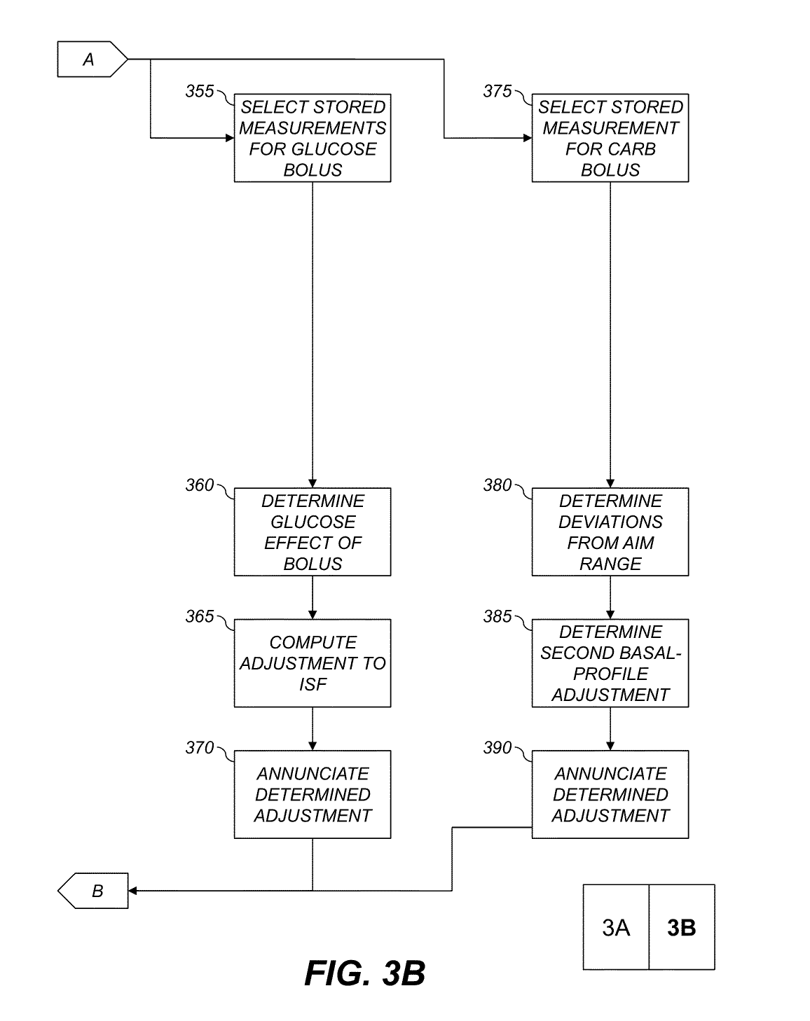

The processor 286 includes one or more data processor(s) that implement processes of various embodiments described herein, e.g., embodiments discussed above and methods shown in FIGS. 3A-3B, discussed below. A “data processor” is a device for processing data and can include a central processing unit (CPU), a desktop computer, a laptop computer, a mainframe computer, a personal digital assistant, a digital camera, a cellular phone, a smartphone, or any other device for processing data, managing data, or handling data, whether implemented with electrical, magnetic, optical, biological components, or otherwise. The phrase “communicatively connected” includes any type of connection, wired or wireless, between devices, data processors, or programs in which data can be communicated. Subsystems such as the peripheral system 220, the user interface 230, and the storage device 240 are shown separately from the processor 286 but can be stored completely or partially within the processor 286.

The storage device 240 includes or is communicatively connected with one or more tangible non-transitory computer-readable storage medium(s) configured to store information, including the information needed to execute processes according to various embodiments. The term “device” does not imply that storage device 240 include only one piece of hardware that stores data. A “tangible non-transitory computer-readable storage medium” as used herein refers to any non-transitory device or article of manufacture that participates in storing instructions which may be provided to the processor 286 for execution. Such a non-transitory medium can be non-volatile or volatile. Examples of non-volatile media include floppy disks, flexible disks, or other portable computer diskettes, hard disks, magnetic tape or other magnetic media, Compact Discs and compact-disc read-only memory (CD-ROM), DVDs, BLU-RAY disks, HD-DVD disks, other optical storage media, Flash memories, read-only memories (ROM), and erasable programmable read-only memories (EPROM or EEPROM). Examples of volatile media include dynamic memory, such as registers and random access memories (RAM).

Computer program instructions are read into the memory 241 from the disk 242, or a wireless, wired, optical fiber, or other connection. The processor 286 then executes one or more sequences of the computer program instructions loaded into the memory 241, as a result performing process steps and other processing described herein. In this way, the processor 286 carries out a computer implemented process that provides technical effects described herein. For example, blocks of the flowchart illustrations or block diagrams herein, and combinations of those, can be implemented by computer program instructions.

In various embodiments, the processor 286 is communicatively connected to a communication interface 215 that is coupled via a network link 216 to the network 116. For example, the communication interface 215 can be a WIFI or BLUETOOTH SMART wireless transceiver and the network link 216 can be a radio-frequency (RF) communications channel. As another example, the communication interface 215 can be a network card to provide a data communication connection to a compatible local-area network (LAN), e.g., an Ethernet LAN, or wide-area network (WAN). The communication interface 215 sends and receives electrical, electromagnetic or optical signals that carry digital data streams representing various types of information across the network link 216 to the network 116. The network link 216 can be connected to the network 116 via a switch, gateway, hub, router, or other networking device.

The processor 286 can send messages and receive data, including program code, to and from the network 116 via the network link 216 and the communication interface 215. For example, requested code for an application program (e.g., a JAVA applet or smartphone app) can be stored on a tangible non-volatile computer-readable storage medium connected to the network 116. A network server (not shown) can retrieve the code from the medium and transmit it via the network 116 to the communication interface 215. The received code can be executed by the processor 286 as it is received, or stored in the storage device 240 for later execution.

Moreover, program code to carry out methods described herein can execute entirely on a single processor 286 or on multiple communicatively-connected processors 286. For example, code can execute wholly or partly on a user's computer and wholly or partly on a remote computer, e.g., a server. The remote computer can be connected to the user's computer through the network 116. The user's computer or the remote computer can be non-portable computers, such as conventional desktop personal computers (PCs), or can be portable computers such as tablets, cellular telephones, smartphones, or laptops.

Embodiments of the present invention can take the form of computer program products embodied in one or more tangible non-transitory computer readable medium(s) having computer readable program code embodied thereon. Such medium(s) can be manufactured as is conventional for such articles, e.g., by pressing a CD-ROM. The program(s) embodied in the medium(s) include computer program instructions that can direct the processor 286 to perform a particular series of operational steps when loaded, thereby implementing functions or acts specified herein.

FIGS. 3A-3B are a flowchart illustrating exemplary methods for recommending adjustments. For example, illustrated is a method for recommending a basal-rate adjustment for an insulin-delivery system. For clarity of explanation, reference is herein made to various components shown in FIGS. 1 and 2 that can carry out or participate in the steps of the exemplary method. Accordingly, the method can include automatically performing steps described herein using the processor 286, FIG. 2. It should be noted, however, that other components can be used; that is, the exemplary method is not limited to being carried out by the identified components. For purposes of this exemplary embodiment, processing begins with step 305.

In step 305, a physiological parameter of a patient is continuously measured. As described above, “continuous” measurement can be recurring, e.g., every 5 minutes. The physiological parameter can be, e.g., blood glucose. Step 305 can be followed by step 310 or step 335.

In step 310, the patient is infused with insulin according to an initial basal profile and the continuous physiological parameter measurements.

In step 315, historical data of the delivery of insulin are stored. The next step can be step 340 or step 310. In this way, the patient is repeatedly infused with insulin. Steps 310, 320, and 330 can be repeated in any order or combination, and any number of times.

In step 320, using the processor 286, deviations of the delivery of insulin from the basal profile are automatically determined for one or more time period(s) using the stored historical data. This can be done as described above with reference to FIG. 2.

In step 325, using the processor, a respective first basal-profile adjustment is automatically computed for each of the time period(s) using the determined deviations. This can be done as described above with reference to FIG. 2.

In step 330, using the processor, the computed first basal-profile adjustment(s) is/are automatically annunciates, e.g., via the user interface 230. This can be done as described above with reference to FIG. 2.

In various aspects, the measurements taken in step 310 are provided to step 335. In step 335, using the processor, a plurality of the blood glucose measurements is stored. This can be done as described above with reference to the storage device 240, FIG. 2. Step 335 can be followed by step 355 or step 375.

In step 340, using the processor 286, deviations of blood glucose level from a stored aim range for one or more time period(s) are determined using the stored measurements. This can be done as described above with reference to FIG. 2. As discussed above, the time periods used for glucose-data processing can be different from those used for historical-data processing.

In step 345, respective second basal-profile adjustment(s) are computed for at least some of the time period(s) using the determined deviations. This can be done as described above with reference to FIG. 2. For example, steps 325 or 345 can include performing i or other statistical tests, as described above, as can steps 365 and 385, discussed below.

In step 350, at least some of the computed second basal-profile adjustment(s) is/are annunciated. This can be done as described above with reference to the user interface 230, FIG. 2.

Referring to FIG. 3B, step 355 can follow step 335, FIG. 3A. After the blood glucose measurements for a selected time period are stored in step 335, two stored measurements are selected using the historical data. The two selected measurements correspond to a glucose correction bolus during the selected time period. The measurements can be, e.g., before-bolus and after-bolus measurements. This selection can be done as described above with reference to FIG. 2.

In step 360, using the processor 286, a glucose effect of the glucose correction bolus is determined using the selected stored measurements and a stored aim range. This can be done as described above with reference to FIG. 2.

In step 365, an adjustment to an insulin sensitivity factor is computed for the selected time period using the determined glucose effect. This can be done as described above with reference to FIG. 2.

In step 370, the computed adjustment to the insulin sensitivity factor is annunciated. This can be done as described above with reference to the user interface 230, FIG. 2.

Step 375 can follow step 335, FIG. 3A. After the blood glucose measurements for a selected time period are stored in step 335, at least one of the stored measurements is selected by the processor 286 using the historical data. The at least one selected measurement corresponds to a carbohydrate correction bolus during the selected time period. This can be done as described above with reference to FIG. 2.

In step 380, a respective deviation for each selected stored measurement is automatically determined with respect to a stored aim range. This can be done as described above with reference to FIG. 2.

In step 385, an adjustment to the insulin-carbohydrate (I:C) ratio is computed for the selected time period using the determined deviations and the insulin-carbohydrate ratio. This can be done as described above with reference to FIG. 2.

In step 390, at least some of the computed adjustment(s) to the insulin-carbohydrate ratio is/are annunciated. This can be done as described above with reference to the user interface 230, FIG. 2.

In various aspects, in step 333, a determined adjustment is automatically applied. The adjustment can be an adjustment determined in any of steps 330, 350, 370, or 390. This can be done as described above with reference to FIG. 2, e.g., by updating data of the basal profile, ISF, or I:C in the storage device 240.

In a first aspect of a method for recommending a basal-rate adjustment for an insulin-delivery system, steps 305, 310, 315, 320, 325, and 330 are performed in that order. In a second aspect of a method for recommending a basal-rate adjustment for an insulin-delivery system, steps 305, 335, 340, 345, 350 are performed in that order. In a third aspect of a method for recommending an ISF adjustment for an insulin-delivery system, steps 305, 335, 355, 360, 365, 370 are performed in that order. In a fourth aspect of a method for recommending an I:C adjustment for an insulin-delivery system, steps 305, 335, 375, 380, 385, 390 are performed in that order. In various aspects, one or more of the first through fourth aspects are performed in any combination and in any order. The processor 286 can carry out computation steps of various of the first through fourth aspects interleaved in time, or sequentially. In this way, the first through fourth aspects can each be used independently, or can be used in any combination.

In view of the foregoing, embodiments of the invention provide improved management of data relevant to basal rates and parameters. A technical effect of processing performed by the processor 286 is to compute adjustment recommendations using data provided, e.g., by the measurement device 200, and to compute graphical representations of those recommendations. A further technical effect is to present the graphical representations outside the particular computing device that performed the computations, e.g., to the patient or a healthcare provider who may use the recommendations in determining basal rates or parameters. A further technical effect of various embodiments is to automatically adjust basal rates or parameters to improve control of the patient's blood glucose by the insulin delivery device 102 and the controller 104. Various decision-support systems and devices described herein can be integrated with, e.g., episodic blood glucose meters or drug-delivery devices. Various methods described herein can be performed by processors in such meters or devices.

### PARTS LIST FOR FIGS. 1-3B

100 insulin delivery system
102 insulin delivery device
104 controller
106 infusion set
108 flexible tubing
110 radio frequency (RF) communications link
111 radio frequency (RF) communications link
112 continuous glucose monitoring (CGM) sensor
113 radio frequency (RF) communications link
114 glucose meter
115 test strip
116 network
117 radio frequency (RF) communications link
118 radio frequency communications link
125 test strip
130 housing
144 touchscreen
145 exemplary tape indicator
146 soft key
200 measurement device
215 communication interface
216 network link
220 peripheral system
230 user interface
240 storage device
241 memory
242 disk
286 processor
305, 310, 315, 320, 325 steps
330, 335, 340, 345, 350 steps
355, 360, 365, 370, 375 steps
380, 385, 390 steps
1138 patient

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Separate references to “an embodiment” or “particular embodiments” or the like do not necessarily refer to the same embodiment or embodiments; however, such embodiments are not mutually exclusive, unless so indicated or as are readily apparent to one of skill in the art. The use of singular or plural in referring to “method” or “methods” and the like is not limiting. The word “or” is used in this disclosure in a non-exclusive sense, unless otherwise explicitly noted. To the extent there are variations of the invention that are within the spirit of the disclosure or are equivalent to the inventions found in the claims, it is the intent that this patent will cover those variations as well.

What is claimed is:

1. A decision support system for a patient, the system comprising:
a) a measurement device configured to continuously measure a physiological parameter of the patient;
b) an insulin delivery device configured to provide insulin to the patient according to an initial basal profile and the continuous measurements of the physiological parameter;
c) a storage device holding historical data of insulin delivery to the patient by the insulin delivery device; and
d) a processor coupled to the storage device, the processor being configured to:
i) determine deviations of the delivery of insulin from the basal profile for one or more time period(s) using the historical data;
ii) compute a respective first basal-profile adjustment for each of the one or more time period(s) using the determined deviations; and
iii) annunciate the computed first basal-profile adjustment(s).

2. The system according to claim 1, wherein the one or more time period(s) include a plurality of time periods and the processor is further adapted to:
a) determine whether or not at least one of the plurality of time periods has deviations that are significantly different than an overall deviation of the plurality of time periods using a chi-squared (χ²) test; and
b) determine a single first basal-profile adjustment for at least two different ones of the plurality of time periods if the deviations for the at least one of the plurality of time periods are not significantly different than the overall deviation.

3. The system according to claim 1, wherein the processor is further adapted to adjust the initial basal profile based upon the computed first basal-profile adjustment(s).

4. The system according to claim 1, further including a display, the processor configured to annunciate the computed first basal-profile adjustment(s) by presenting a visual indication thereof on the display.

5. The system according to claim 4, wherein each first basal-profile adjustment includes a respective delivery rate and the visual indication includes textual representation(s) of the respective delivery rate(s).

6. The system according to claim 1, further including a user interface adapted to receive input, the processor further adapted to receive the historical data via the user interface and store the received historical data in the storage device.

7. The system according to claim 1, wherein the historical data includes bolus data and the processor is further configured to filter meal data out of the historical data using the bolus data.

8. The system according to claim 1, wherein the measurement device comprises a continuous glucose monitor and the measured physiological parameter comprises blood glucose.

9. The system according to claim 8, wherein the processor is further configured to store blood glucose measurements of the patient and filter meal data out of the historical data using the stored measurements of the blood glucose.

10. The system according to claim 8, wherein the processor is further configured to:
a) store a plurality of the blood glucose measurements;
b) determine deviations of blood glucose level from a stored aim range for one or more time period(s) using the stored blood glucose measurements;
c) compute a respective second basal-profile adjustment for each of the one or more time period(s) using the determined deviations; and
d) annunciate the respective second basal-profile adjustment(s).

11. The system according to claim 10, wherein the processor is further configured to filter meal data out of the stored blood glucose measurements using the historical data.

12. The system according to claim 10, wherein the processor is configured to determine the deviations of blood glucose level by determining the extent to which each of the stored blood glucose measurements is outside the stored aim range, and determining that the deviation for one of the time period(s) is zero if the stored measurements during that time period are within the stored aim range.

13. The system according to claim 8, wherein the processor is further configured to:
a) store a plurality of the blood glucose measurements for a selected time period;
b) select two stored measurements using the historical data, the two stored measurements corresponding to a glucose correction bolus during the selected time period;
c) determine a glucose effect of the glucose correction bolus using the selected stored measurements and a stored aim range;
d) compute an adjustment to an insulin sensitivity factor for the selected time period using the determined glucose effect; and
e) annunciate the computed adjustment to the insulin sensitivity factor.

14. The system according to claim 8, wherein the storage device holds an insulin-carbohydrate ratio and the processor is further configured to:
a) store a plurality of the blood glucose measurements for a selected time period;
b) select at least one stored measurement using the historical data, the selected at least one stored measurement corresponding to carbohydrate correction boluses during the selected time period;
c) determine a respective deviation for each of the selected stored measurements with respect to a stored aim range;
d) compute an adjustment to the insulin-carbohydrate ratio for the selected time period using the determined deviations and the insulin-carbohydrate ratio; and
e) annunciate the respective adjustment to the insulin-carbohydrate ratio.

15. The system according to claim 14, wherein the storage device further holds a glucose-carbohydrate ratio and the processor is further configured to compute the adjustment to the insulin-carbohydrate ratio using the stored glucose-carbohydrate ratio.

16. A method of recommending a basal-rate adjustment for an insulin-delivery system, the method comprising:
continuously measuring a physiological parameter of a patient;
repeatedly infusing the patient with insulin according to an initial basal profile and the continuous physiological parameter measurements;
storing historical data of the delivery of insulin;
using a processor, automatically determining deviations of the delivery of insulin from the basal profile for one or more time period(s) using the stored historical data;
using the processor, automatically computing a respective first basal-profile adjustment for each of the time period(s) using the determined deviations; and
using the processor, automatically annunciating the computed first basal-profile adjustment(s).

17. The method according to claim 16, wherein the physiological parameter is blood glucose.

18. The method according to claim 17, further including automatically performing the following using the processor:
storing a plurality of the blood glucose measurements;
determining deviations of blood glucose level from a stored aim range for one or more time period(s) using the stored measurements;
computing a respective second basal-profile adjustment for each of the time period(s) using the determined deviations; and
annunciating the computed second basal-profile adjustment(s).

19. The method according to claim 17, further including automatically performing the following using the processor:
storing a plurality of the blood glucose measurements for a selected time period;
selecting two stored measurements using the historical data, the two selected measurements corresponding to a glucose correction bolus during the selected time period;
determining a glucose effect of the glucose correction bolus using the selected stored measurements and a stored aim range;
computing an adjustment to an insulin sensitivity factor for the selected time period using the determined glucose effect; and
annunciating the computed adjustment to the insulin sensitivity factor.

20. The method according to claim 17, further including automatically performing the following using the processor:
storing a plurality of the blood glucose measurements for a selected time period;
selecting at least one of the stored measurements using the historical data, the at least one selected measurement corresponding to carbohydrate correction boluses during the selected time period;
determining a respective deviation for each selected stored measurements with respect to a stored aim range;
computing an adjustment to the insulin-carbohydrate ratio for the selected time period using the determined deviations and the insulin-carbohydrate ratio; and
annunciating the computed adjustment to the insulin-carbohydrate ratio.
